(19) United States
(12) Patent Application Publication
(10) Pub. No.: US 2014/0343395 A1
(43) Pub. Date: Nov. 20, 2014

(54) SYSTEM AND METHOD FOR PROVIDING MAGNETIC BASED NAVIGATION SYSTEM IN DENTAL IMPLANT SURGERY

(71) Applicants: Woncheol Choi, San Jose, CA (US); Jia Leung Mar, Union City, CA (US)

(72) Inventors: Woncheol Choi, San Jose, CA (US); Jia Leung Mar, Union City, CA (US)

(73) Assignee: Anatomage Inc., San Jose, CA (US)

(21) Appl. No.: 13/895,222

(22) Filed: May 15, 2013

(51) Int. Cl. A61B 5/06

(52) U.S. Cl. CPC A61B 5/062
USPC 600/409

(57) ABSTRACT

A system and method for providing magnetic based navigation in dental implant surgery is disclosed. According to one embodiment, a surgical navigation system comprises a custom piece that is made to fit in a patient's anatomy and comprises a first magnetic component. The surgical navigation system further comprises an instrument assembly comprising a drill bit and a second magnetic component. A processing unit receives and displays a first position and orientation data from the first magnetic component with respect to a second position and orientation data from the second magnetic component to track the movement of the first magnetic component with respect to the second magnetic component.

### FIELD

The field of the invention relates generally to dental implant surgery, and more particularly to magnetic based navigation system in dental implant surgery.

### BACKGROUND

With increasing use of CT scans in dentistry, dental implant surgery is becoming computer-guided surgery. In a typical computer-guided surgery, a doctor reviews a patient's CT scan and plans a surgical procedure in specialized software. After the surgical plan is complete, the doctor sends the plan data to a surgical guide company, and the surgical guide company sends back a surgical guide that follows the doctor's plan. The surgical guide is fabricated to custom fit to the patient and has a mechanical mechanism guiding a drill. By drilling with the surgical guide, the doctor can accomplish the surgery with great accuracy as planned on the software. This computer-guided surgery has a significant improvement over traditional dental surgery with no guiding capability to match the plan in software.

A surgical navigation system uses a tracking marker that is attached to the drill piece and another tracking marker that is attached onto the patient's mouth or jawbone. The tracking markers emit a signal that an external sensor acquires. The system may also have an external device that emits a signal to the tracking markers, and subsequently the tracking markers relay a modified signal to the external sensor. The external sensor sends data to a computer running navigation software. The navigation software processes the data, for example, using a triangulation algorithm, to calculate the three-dimensional (3D) coordinates of the tracking markers on the drill piece and patient's mouth. The navigation software then shows real-time positioning of the drill piece with respect to the virtual implant plan. The navigation system tracks the position of the instrument in reference to the patient dentition and sends visual feedback to doctor in real time. The visual feedback allows doctors to achieve precisely identical surgery as planned on a computer.

Traditional navigation systems require optical trackers that are attached to both instruments and a patient's dentition. This configuration creates challenges in surgery. The line of sight has to be established between tracking markers and a sensor. This makes doctor's operation difficult especially when the instrument is positioned to drill a hole in a specific angle inside the patient's mouth. Bulky optical trackers make it difficult to manipulate the instrument during a surgical procedure within a tight space inside the patient's mouth. Furthermore, optical trackers have intrinsic line of sight issues. Any obstructions between optical markers, a signal emitter, or a sensor hinder the accuracy. When the doctor or the instrument blocks optical markers during a surgery, the tracking of the instrument is lost. Also, the external sensor and signal emitter are typically large and takes up significant space within the vicinity of the doctor and patient. Resultantly, doctors have a limited space to operate surgery due to the bulkiness of the tracking device within the patient's mouth and equipment nearby.

Recent advancements allow for the sensors to be smaller in size and to have better data acquisition. The sensor of a navigation system is made smaller and fitted on to the dental drill piece. The tracking markers can also be made smaller, while the signal acquired by the sensor is sufficient in accuracy.

For dental surgery, the size of instrument is very important. Doctor has to maneuver the instrument inside a patient's mouth where the space is very confined. Also, teeth, soft tissue, tongue are lightly located, making delicate maneuvering of the instrument difficult.

### SUMMARY

A system and method for providing magnetic based navigation in dental implant surgery is disclosed. According to one embodiment, a surgical navigation system comprises a custom piece that is made to fit in a patient's anatomy and comprises a first magnetic component. The surgical navigation system further comprises an instrument assembly comprising a drill bit and a second magnetic component. A processing unit receives and displays a first position and orientation data from the first magnetic component with respect to a second position and orientation data from the second magnetic component to track the movement of the first magnetic component with respect to the second magnetic component.

The above and other preferred features, including various novel details of implementation and combination of elements, will now be more particularly described with reference to the accompanying drawings and pointed out in the claims. It will be understood that the particular methods and apparatuses are shown by way of illustration only and not as limitations. As will be understood by those skilled in the art, the principles and features explained, herein may be employed in various and numerous embodiments.

### BRIEF DESCRIPTION OF THE DRAWINGS

The accompanying drawings, which are included as part of the present specification, illustrate the presently preferred embodiment of the present invention and together with the general description given above and the detailed description of the preferred embodiment given below serve to explain and teach the principles of the present invention.

FIG. 1 illustrates an exemplary magnetic-based tracking system, according to one embodiment;

FIG. 2 illustrates an exemplary anatomical image of a patient's CT scan rendered in planning software, according to one embodiment;

FIG. 3 illustrates an exemplary virtual mouthpiece assembly, according to one embodiment;

FIG. 4 illustrates an exemplary physical mouthpiece assembly, according to one embodiment;

FIG. 5 illustrates an exemplary instrument assembly, according to one embodiment;

FIGS. 6a and 6b illustrate an exemplary coordinate transformation between a transmitter's coordinate system and a patient CT scan coordinate system, according to one embodiment;

FIG. 7 illustrates an exemplary navigated surgical procedure, according to one embodiment;

FIGS. 8a and 8b illustrate an exemplary procedure for registering drill dimensions using an apparatus, according to one embodiment; and

FIG. 9a and 9b illustrate an exemplary calibration process using a calibration feature on a mouthpiece, according to one embodiment.

It should be noted that the figures are not necessarily drawn to scale and that elements of structures or functions are generally represented by reference numerals for illustrative purposes throughout the figures. It also should be noted that the figures are only intended to facilitate the description of the various embodiments described herein. The figures do not describe every aspect of the teachings described herein and do not limit the scope of the claims.

### DETAILED DESCRIPTION

A system and method for providing magnetic based navigation in dental implant surgery is disclosed. According to one embodiment, a surgical navigation system comprises a custom piece that is made to fit in a patient's anatomy and comprises a first magnetic component. The surgical navigation system further comprises an instrument assembly comprising a drill bit and a second magnetic component. A processing unit receives and displays a first position and orientation data from the first magnetic component with respect to a second position and orientation data from the second magnetic component to track the movement of the first magnetic component with respect to the second magnetic component.

In the following description, for purposes of clarity and conciseness of the description, not all of the numerous components shown in the schematic are described. The numerous components are shown in the drawings to provide a person of ordinary skill in the art a thorough enabling disclosure of the present invention. The operation of many of the components would be understood to one skilled in the art.

Each of the additional features and teachings disclosed herein can be utilized separately or in conjunction with other features and teachings to provide the present table game. Representative examples utilizing many of these additional features and teachings, both separately and in combination, are described in further detail with reference to the attached drawings. This detailed description is merely intended to teach a person of skill in the art further details for practicing preferred aspects of the present teachings and is not intended to limit the scope of the claims. Therefore, combinations of features disclosed in the following detailed description may not be necessary to practice the teachings in the broadest sense and are instead taught merely to describe particularly representative examples of the present teachings.

The methods presented herein are not inherently related to any particular computer or other apparatus. Various general-purpose systems may be used with programs in accordance with the teachings herein, or it may prove convenient to construct more specialized apparatus to perform the required method steps. The required structure for a variety of these systems will appear from the description below. In addition, the present invention is not described with reference to any particular programming language. It will be appreciated that a variety of programming languages may be used to implement the teachings of the invention as described herein.

Moreover, the various features of the representative examples and the dependent claims may be combined in ways that are not specifically and explicitly enumerated in order to provide additional useful embodiments of the present teachings. In addition, it is expressly noted that all features disclosed in the description and/or the claims are intended to be disclosed separately and independently from each other for the purpose of original disclosure, as well as for the purpose of restricting the claimed subject matter independent of the compositions of the features in the embodiments and/or the claims. It is also expressly noted that all value ranges or indications of groups of entities disclose every possible intermediate value or intermediate entity for the purpose of original disclosure, as well as for the purpose of restricting the claimed subject matter. It is also expressly noted that the dimensions and the shapes of the components shown in the figures are designed to help understand how the present teachings are practiced but are not intended to limit the dimensions and the shapes shown in the examples.

CT-based dental 3D imaging is more widely available. Dental implant surgery utilizing dental CT scan is very helpful for achieving successful surgery. It can aid in treatment planning and clinically sound placement of implants and to avoid major critical structure such as inferior alveolar nerve or sinus.

In planning a dental implant, a doctor creates a virtual implant plan based on the CT image data of a patient. Based on the virtual implant plan, a computer-aided designed (CAD) mouthpiece is fabricated to fit onto the patient's mouth. According to one embodiment, a mouthpiece is custom fabricated with at least one tracking marker, usually optically-based. The tracking marker's position on the mouthpiece is already known at time of design from a virtual implant plan. According to one embodiment, a drill piece fitted with one or more tracking markers is provided. The fabricated mouthpiece is positioned on the patient's mouth in the same position as it was designed from in the virtual implant plan.

The tracking markers emit a signal that an external sensor acquires. According to one embodiment, the present system has an external device that emits a signal to the tracking markers. The tracking markers receive the signal and relay a modified signal including their position and orientation information to the external sensor. The external sensor sends data to a computer running navigation software. The navigation software processes the received data, for example, using a triangulation algorithm, to calculate the 3D coordinates of the tracking markers. In one embodiment, the present system has specialized processing hardware that calculates and sends 3D coordinate data to the navigation software. According to one embodiment, navigation software provides real-time visual feedback of the drill piece with respect to the virtual implant plan. The navigation software tracks the position of the instrument in reference to the patient's anatomy. The navigation software displays the drill piece in its actual position and orientation in real-time relative to the implant position in the virtual implant plan.

According to one embodiment, a plurality of magnetic components systematically positioned with respect to the patient's anatomy. The plurality of magnetic components includes at least one magnetic signal transmitter and one or more magnetic sensors. A processing unit receives data and calculates the position and orientation of the magnetic sensors with respect to the magnetic signal transmitter. The navigation software receives the position and orientation data of the magnetic sensors and provides a real-time visual feedback to the doctor by tracking the magnetic sensors with respect to the magnetic signal transmitter that is affixed to the patient anatomy.

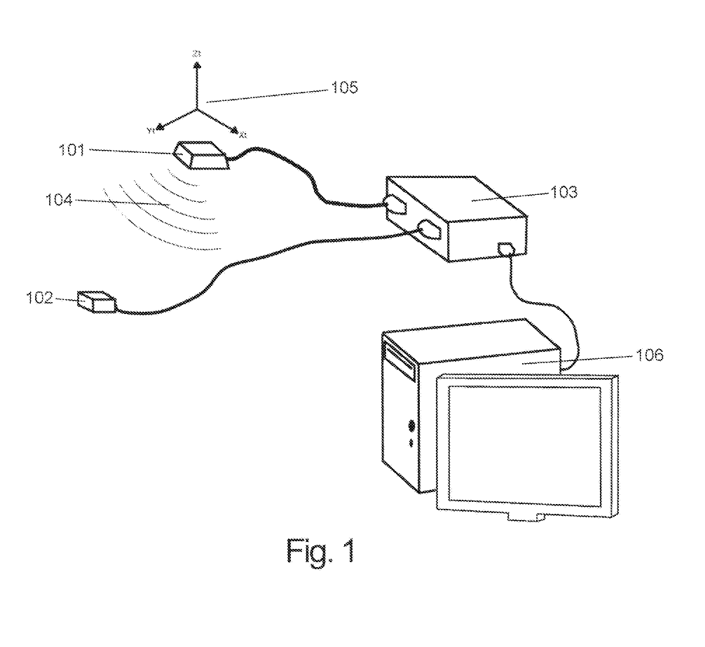

The present system and method implements magnetic based tracking. Magnetic based tracking is advantageous to optical based to overcome the line of sight issue during surgery. FIG. 1 illustrates an exemplary magnetic-based tracking system, according to one embodiment. The magnetic-based tracking system 100 has a transmitter 101, a sensor 102, and a processing unit 103. The transmitter 101 has a fixed shape of known dimensions known to the system and emits a magnetic field signal 104. The transmitter 101 is connected to the processing unit 103 by a cable or wirelessly. The transmitter has its own coordinate system 105 with primary axes aligned with its external dimensions.

The sensor 102 detects the magnetic field signals emitted from the transmitter 101, and generates data based on detection of the magnetic field signals. The sensor 102 is connected to the system's processing unit 103 by a cable, or wirelessly and sends the data received from the transmitter 101 or processes the data and sends the position and orientation data to the processing unit 103 via a known communication protocol. The sensor 102 has a fixed shape of known dimensions, and the center position of the sensor is fixed and known to the system.

According to one embodiment, the magnetic-based tracking system 100 is Fastrak® manufactured by Polhemus of Colchester. Vt. The magnetic-based tracking system 100 uses electro-magnetic fields to determine the position and orientation of a remote object using one or more sensors 102 attached to the remote object. The transmitter 101 generates near field, low frequency magnetic field vectors from a single assembly of three concentric, stationary antennas. The sensor 102 detects the field vectors with a single assembly of three concentric, remote sensing antennas. The sensed signals are input to a mathematical algorithm that computes the sensor 102's position and orientation relative to the transmitter 101. According to one embodiment, the magnetic-based tracking system 100 is capable of operating multiple sensors 102 at discrete carrier frequencies. Different carrier frequencies allow the operation of multiple sensors 102 simultaneously and in close proximity to one another. The processing unit 103 interfaces to a host computer 106 via RS-232 and USB serial communication or wirelessly.

The processing unit 103 tracks the position and orientation of the sensor 102 up to 6 degrees of freedom in real time. This sensor position and orientation is represented in three-dimensional Cartesian coordinates (x, y, z) and orientation angles (azimuth, elevation, roll) in the transmitter's coordinate system 105. The processing unit 103 sends the sensor position and orientation data to the host computer 106. The sensor 102 must be within a close proximity (e.g., 5 to 8 inches) to the transmitter 101 to receive the transmitted signal without significant signal degradation. The further the sensor 102 is from transmitter 101, it will be less accurate or cannot be tracked if it is too far from the transmitter 101. The magnetic-based tracking system 100 has a sufficient sampling rate (e.g., 30 to 60 Hz) to provide data samples to the host computer 106 in real time such that there is little to no lag in tracking. The magnetic-based tracking system 100 achieves the accuracy of 0.03 inches RMS with a resolution of 0.0002 inches per inch. This accuracy is good enough for providing a doctor visual tracking and feedback of a mouthpiece and drilling equipment during a dental surgery.

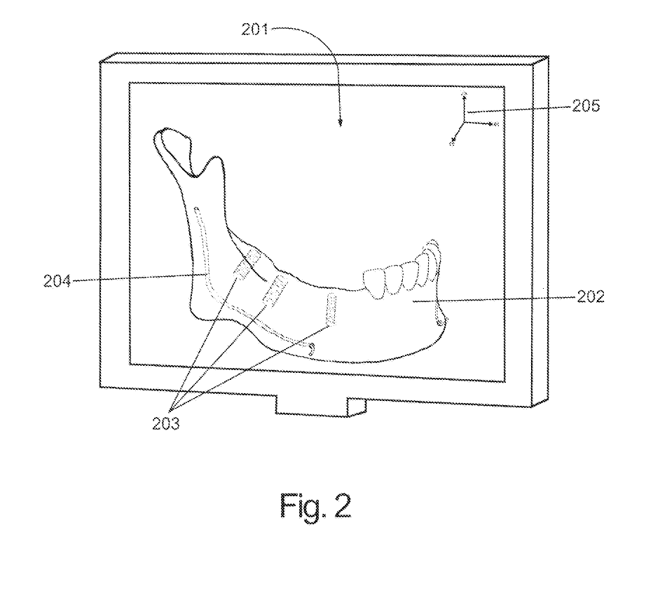

FIG. 2 illustrates an exemplary anatomical image of a patient's CT scan rendered in planning software, according to one embodiment. A patient's anatomical image 202 such as CT scan image is rendered in implant planning software 201 on a computer display of the host computer 106. The planning software 201 allows the doctor to visualize the patient's anatomy in a virtual space and create a virtual implant plan. In the virtual implant plan, the doctor decides to places one or more virtual implant models 203 at desired locations within patient's anatomy. The planning software 201 also renders other objects 204 such as abutment, restoration, nerve mapping to show the spatial relationship between virtual implant models 203 and other objects 204 with the patient's anatomy. This establishes the three-dimensional coordinates of the virtual implants 203 and objects 204 with respect to the CT coordinate system 205. Information such as trajectory, distances between objects, vital anatomical structures is calculated and displayed to the doctor.

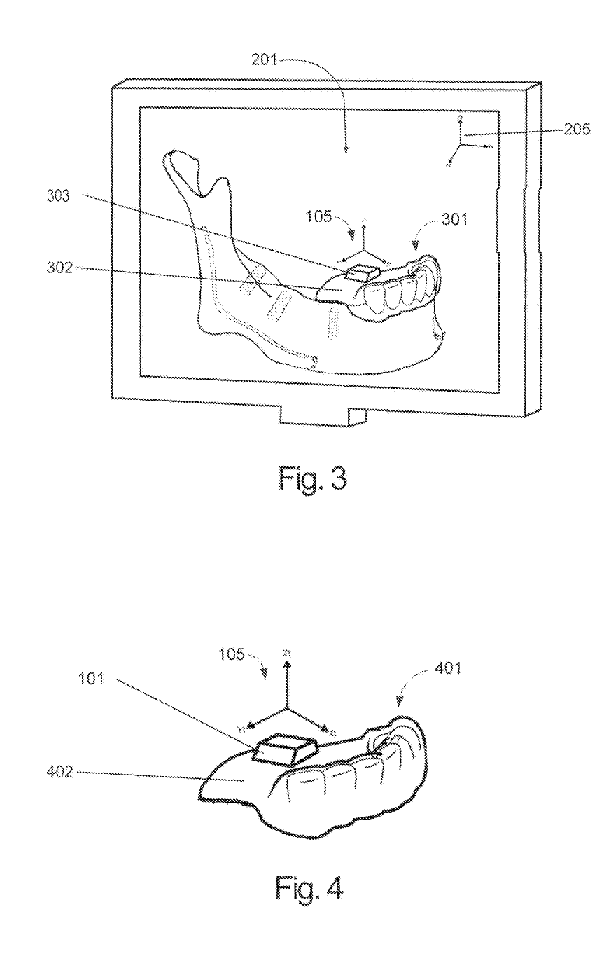

FIG. 3 illustrates an exemplary virtual mouthpiece assembly, according to one embodiment. A virtual mouthpiece assembly 301 has a virtual mouthpiece 302 customized to fit to a patient, and a virtual transmitter 303 attached to the virtual mouthpiece 302. The user can position the virtual transmitter 303 in the computer model to finalize the location and coordinate system 105 of the virtual transmitter 303.

Using the planning software, the customized virtual mouthpiece 302 is designed from a patient CT scan or a dentition surface model of the patient. The dentition surface model may be acquired by a CT or optical scan of the patient's impression, a stone model, a direct intra-oral scan, any combination of a CT, optical, intra-oral scan, or any other technique known in the art. According to one embodiment, the mouthpiece is fabricated with the intra-oral scan data merged to the patient's CT data. Based on the combination of CT and surface information, or CT information alone, the virtual mouthpiece 302 is fabricated to fit specifically onto the patient's mouth. In one embodiment, the virtual mouthpiece 302 is designed based on the three-dimensional digital inversion, a conventional lab based technique, or CAD/CAM system. The virtual mouthpiece 302 has a fixed position in the patient CT, the same position a physical mouthpiece will seat onto the real patient. Thus, the virtual mouthpiece 302 is represented in the patient CT coordinate system 205. The intended position of the virtual transmitter 303 attachment on the virtual mouthpiece 302 is designed virtually. The transmitter's coordinate system 105 is determined within the patient CT coordinate system 205 by coordinate transformation. The coordinates of design features of the virtual mouthpiece 302 is determined within the patient CT coordinate system 205. Using the virtual mouthpiece assembly 301 created from the planning software, a physical mouthpiece assembly is created as illustrated in FIG. 4.

FIG. 4 illustrates an exemplary physical mouthpiece assembly, according to one embodiment. A physical mouthpiece assembly 401 has a physical mouthpiece 402 and a transmitter 101. The physical mouthpiece 402 may be fabricated based on the virtual mouthpiece 302 created by the planning software using a 3D printer, a CAD/CAM system or through a conventional lab technique. The physical mouthpiece 402 has the attachment for a physical transmitter 101.

The position of transmitter 101 on the physical mouthpiece 402 is known. In one embodiment, the transmitter 101 has a cable extending to the processing unit 103, which is connected to the host computer 106. In another embodiment, the transmitter 101 transmits signals to the processing unit 103 wirelessly.

The transmitter 101 has its own coordinate system 105. With the transmitter 101 attached to the physical mouthpiece assembly 401 at a fixed point, the physical mouthpiece 402 is fixed within the transmitter's coordinate system 105. Since the virtual mouthpiece 302 and the transmitter 303 has a fixed position in the patient's CT scan, and the physical mouthpiece 402 and transmitter 101 is consistent with the virtual mouthpiece 302 and transmitter 303, the physical transmitter's coordinate system 105 can be made consistent to the patient CT coordinate system 205. The position and orientation of nearby sensors 102 connected to the system can now be tracked relative to the transmitter 101 and physical mouthpiece 402.

If a sensor 102, instead of a transmitter 101, is integrated in the physical mouthpiece assembly 401 at a fixed point, the mouthpiece's position and orientation becomes trackable within the nearby transmitter's coordinate system 105 in a rigid body motion. The mouthpiece's position and orientation can be represented within the nearby transmitter's coordinate system 105. Since the virtual mouthpiece 302 and sensor 304 has a fixed position in the patient CT scan, and the physical mouthpiece 402 and sensor 102 is consistent with the virtual mouthpiece 302 and sensor 304, the nearby transmitter coordinate system 105 can be made consistent with patient's CT coordinate system 205.

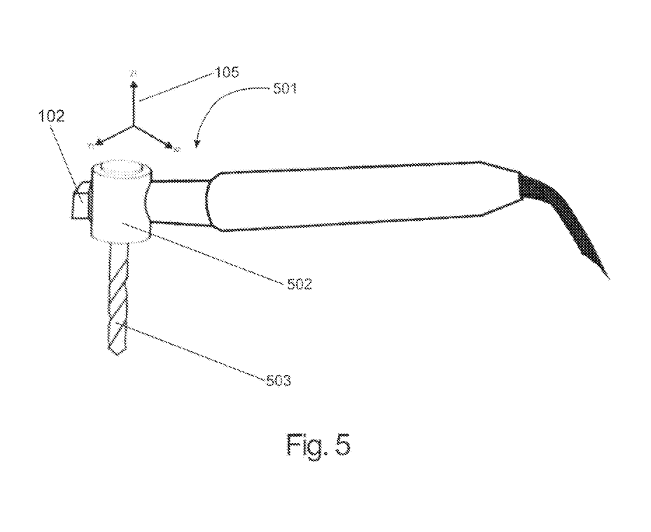

FIG. 5 illustrates an exemplary instrument assembly, according to one embodiment. The instrument assembly 501 has a drilling hand piece 502 and a sensor 102 attached to the hand piece 502. The position of the sensor 102 on the hand piece 502 is known to the system. The sensor 102 can be attached or integrated to the hand piece 502 as a part of the hand piece housing. In one embodiment, the sensor 102 is connected to the processing unit 103 using a data cable. In another embodiment, sensor 102 is connected to the processing unit 103 wirelessly. The processing unit 103 is connected to the host computer 106 and processes the six degrees of freedom position and orientation of the sensor 102 in real time. If connected wiredly, the cable is tied to the hand piece driver cord or integrated to the hand piece cord. The drill hand piece 502 accepts drill bits 503 of different diameters and lengths for drilling a hole at a planned implant site of the patient.

Because the sensor 102 is incorporated in the instrument assembly at a fixed point, the instrument's position and orientation becomes trackable within the nearby transmitter's coordinate system 105 in a rigid body motion. The position and orientation of the instrument assembly 501 can be represented within the nearby transmitter's coordinate system 105. The transmitter coordinate system 105 is made consistent with the patient CT coordinate system 205, therefore the position and orientation of the drill piece 502 can be tracked relative to the patient CT coordinate system 205.

According to one embodiment, a transmitter 101 is incorporated in the instrument assembly 501 instead of a sensor 102. In this case, a sensor 102 may be incorporated in the mouthpiece 402. The pair of the transmitter 101 and the sensor 102 is used to track the instrument assembly 501 in a reverse tracking. The transmitter 101 is incorporated in the instrument assembly 501 at a fixed point, the instrument is fixed within the transmitter 101's coordinate system 105. The position and orientation of nearby sensors connected to the system can be tracked relative to the instrument assembly 501. Since the mouthpiece 402 (and its sensor 102) has a fixed position in the patient CT scan, the patient CT coordinate system 205 can be made consistent to the transmitter coordinate system 105, or vice versa.

According to another embodiment the transmitter 101 is attached to neither mouthpiece assembly 401 nor instrument assembly 501 but to another object within the detectable range of sensors 102. In this case, a sensor 102 is attached to both the mouthpiece assembly 401 and the instrument assembly 501. In this manner, it is possible to track the movement of the mouthpiece 402. The transmitter's coordinate system 105 is made consistent to the patient CT coordinate system 205 through a known systematic relation.

Although the present example of FIGS. 3-5 shows a particular embodiment of the transmitter 101 integrated onto the mouthpiece 402 and the sensor 102 integrated onto the drill piece 502, it is understood that the physical transmitter 101 and the sensor 102 can be integrated onto other objects within a detectable range. The pair of virtual transmitter 303 and sensor 302 tracks the movement of a drill piece with respect to the system's coordinate system during a surgery to provide visual feedback to the doctor.

In the present magnetic-based tracking system 100, the transmitter 101 establishes the origin of the coordinate system 105. The transmitter 101 emits magnetic field signals in the proximate area of one or more sensor 102, but it does not send its own position data to the processing unit 103. If the transmitter 101 moves, its movement is undetected by the system and the system processes the movement as if the sensor 102 moved.

The physical mouthpiece 402 is to be placed in the patient's mouth in the same position as it was predefined in the patient's virtual plan as shown in FIG. 3. Any initial placement or movement error of the physical mouthpiece 402 can induce an undetectable error to the system.

According to one embodiment, to detect or reduce a mouthpiece placement error, an auxiliary device is used to compare the placement of the physical mouthpiece 402 to a known reference point known to the system. In one embodiment, a bite registration device is used. The bite registration device may have mechanical mating features to precisely control placement of the mouthpiece. The bite registration device may also have multiple registration features to precisely compensate a placement error. The present system and method limits a movement error of the mouthpiece. For example, the mouthpiece 402 is locked down through one or more fixation pins that hold it in an established position in patients mouth. The fixation pins are placed in multiple spots that go through the mouthpiece into the soft tissue or the bone of the patient. The fixation pins prevent movement errors introduced after mouthpiece placement and allows for precise registration between the virtual mouthpiece shown in relation to the virtual objects and the physical mouthpiece in the patient's mouth.

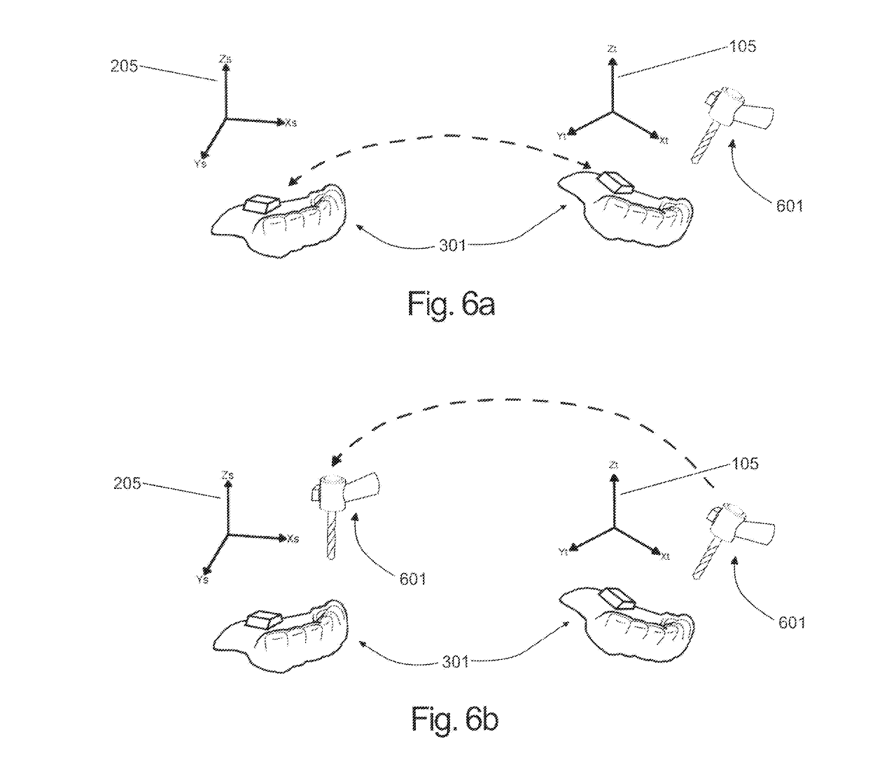

FIGS. 6a and 6b illustrate an exemplary coordinate transformation between a transmitter's coordinate system and a patient CT scan coordinate system, according to one embodiment. The three-dimensional coordinate of a point or an object in the patient's image can be represented in any coordinate system using a rigid body transformation matrix. For example, anatomical structure and other virtual objects such as implants in the patient's image can be transformed into the coordinate system of the transmitter, or vice versa. In one embodiment, the patient CT coordinate system 205 is used to track the drill piece during a surgery. In this case, any object represented in the transmitter's coordinate system 105 is transformed into the patient CT coordinate system 205. Any physical object in a real space registered with respect to any coordinate system can be represented by the three-dimensional coordinate as long as the relationship or the transformation matrix is known.

The transmitter's coordinate system 105 is defined by the transmitter 101. The patient CT coordinate system 205 established in CT scan data is defined by the CT scan imaging software. Regardless of the transmitter 101's location or method of attachment to a mouthpiece or instrument, the mouthpiece and instrument's position and orientation are defined in the transmitter's coordinate system 105. The position and orientation of the mouthpiece and instrument are transformed into the patient CT coordinate system.

The physical mouthpiece 402 mates to the patient's mouth in the same fixed position and orientation as the virtual mouthpiece 302 that was designed in the patient CT scan within planning software. Since the virtual mouthpiece 302 has known position and orientation in both the patient CT coordinate system 205 and the transmitter's coordinate system 105, one coordinate system can be transformed into the other coordinate system through coordinate system transformation. The static anatomical structure of the patient's mouth or objects in the patient CT scan can be represented within the transmitter's coordinate system 105. Likewise, the virtual mouthpiece assembly and virtual instrument assembly 601 within the transmitter coordinate system can be represented within the patient CT coordinate system 205.

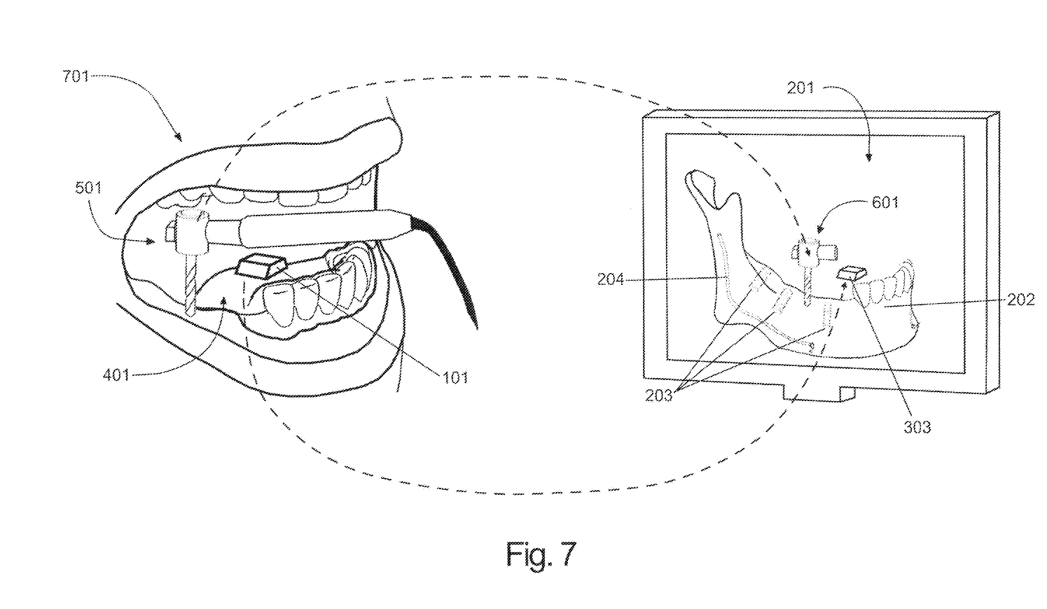

FIG. 7 illustrates an exemplary navigated surgical procedure, according to one embodiment. A processing unit receives input data from the sensor of the instrument assembly 501 that is trackable within the transmitter's coordinate system. The processing unit calculates position and orientation data and sends the data to the computer for further data processing and visualization on the display showing planning software 201 for the doctor's visual feedback. The planning software 201 knows both the fixed position and orientation the virtual mouthpiece assembly 301 in the patient CT coordinate system 205 and the position and orientation of the physical mouthpiece assembly 401 in the transmitter's coordinate system 105, therefore the planning software 201 is able to transform the patient CT coordinate system 205 into the transmitter's coordinate system 105, or vice versa. This allows for the visual representation of patient's anatomy and other virtual objects in either one of the representative coordinate systems.

The physical mouthpiece assembly 401 placed in the patient's mouth 701 at the intended position and orientation. The system is constantly updated on the display showing planning software 201 to provide the doctor with a real time feedback of the instrument and drill position and orientation with respect to the patient's anatomy 202, implants 203, and/or any other reference point. This provides the doctor with a capability to visually track the position of the physical instrument assembly 501 in the patient CT coordinate system 205. As the physical instrument assembly 501 moves during a surgery, the position and orientation of the virtual instrument assembly 601 are updated in real time on the computer display showing planning software 201. During a surgery, if the accuracy of the instrument tracking is determined to be unreliable due to various factors, for example, the sensor 102 moves out of the transmitter 101's range of reliable tracking, magnetic field interference, or unreliable tracking data, the planning software 201 displays a warning message to inform the doctor. In one embodiment, the planning software calculates and displays on the planning software 201 useful information for the doctor during the surgery such as the position and orientation offsets, an error to the intended drill trajectory while drilling, a distance to a vital structure in the patient's anatomy.

According to one embodiment, the physical instrument assembly 501 has a feature that relays a feedback from the planning software, so the doctor does not have to look at the display showing planning software 201 and keeps his vision on the surgery. For example, the physical instrument assembly 501 has a visual indicator, such as an LED light or small LCD panel. The visual indicator provides the doctor with information such as drilling accuracy being within a tolerance or simply a warning if drill is too close to a vital anatomy of the patient. In another embodiment, the physical instrument assembly 501 has an audible feedback that provides an audible sound or haptic feedback that provides vibration or a tactile feedback to inform the doctor regarding the drilling accuracy or if drill is too close to vital anatomy of the patient.

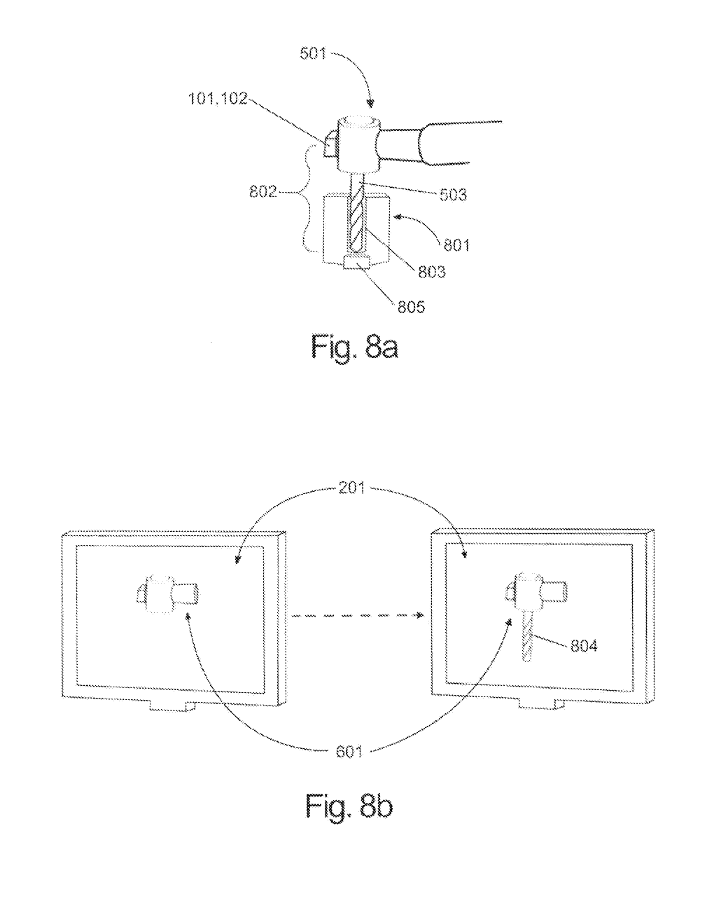

FIGS. 8a and 8b illustrate an exemplary procedure for registering drill dimensions using an apparatus, according to one embodiment. For a physical instrument assembly 501, a drill offset 802 from the sensor 102 to the tip of an attached drill bit 503 must be determined to register the drill position and orientation within the transmitter's coordinate system. According to one embodiment, a registration apparatus 801 of known dimensions is used to register the drill offset 802. A drill bit 503 of the instrument assembly 501 is inserted into the socket 803 of the registration apparatus 801. Another sensor 805 is attached to the registration apparatus 801 with a known position offset. The socket 803 has a physical stop that makes contact, with the drill bit 503 when it is fully inserted. In this static position, the drill 503's critical features (e.g., tip length, position and orientation) are determined from the position and orientation data from the sensors 102 and geometric calculation. During a surgery, the drill bit 503 may be changed several times, and each drill bit may have a different length and size. The planning software supports this drill registration to determine the drill shape whenever a new drill bit is used. This allows the virtual instrument assembly 601 to be accurately updated and the drill bit information is updated on the planning software 201 accordingly. The present method of drill bit registration using a registration apparatus allows for more accurate and faster exchange of drill bits than manually measuring the offset with a measuring tool and entering the measured value into the planning software during registration. Instead of the sensor 102, a single transmitter 101 may be used instead in apparatus 801 or instrument assembly 501 to accurately determine the drill offset 802. It is also understood that the registration of an instrument assembly can be performed in a variety of ways using a sensor, a transmitter, and/or a combination of a sensor and a transmitter in a similar way as described above without deviating the scope of the present subject matter.

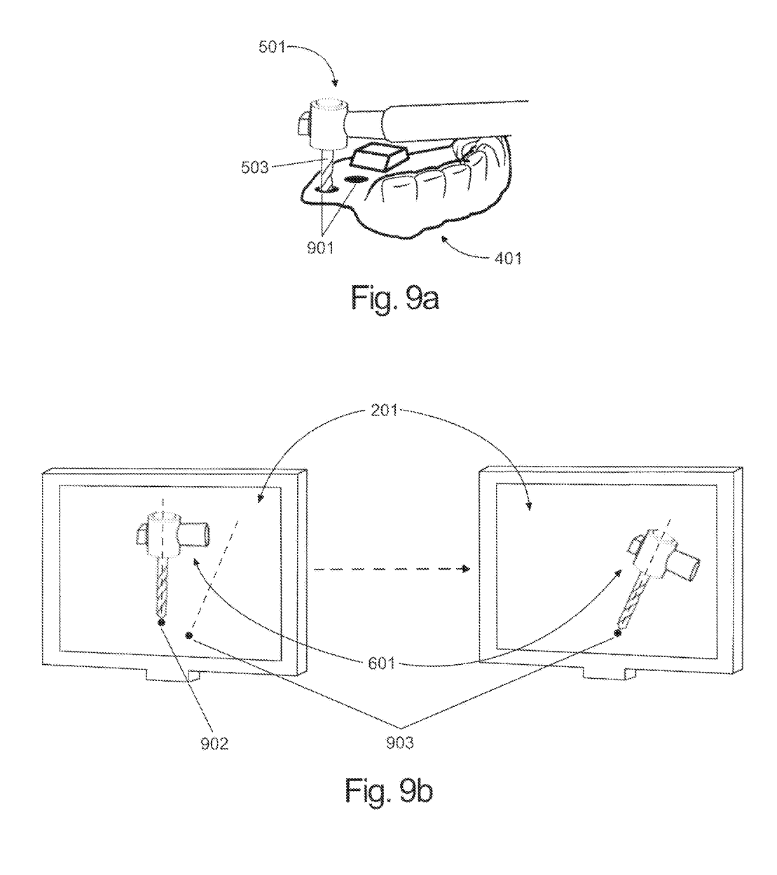

Instrument tracking accuracy may be off due to various factors such as magnetic field interference, an incorrect or unstable position of a mouthpiece, etc. The present navigation system may provide additional assurance to the doctor that the current instrument tracking is accurate. FIGS. 9a and 9b illustrate an exemplary calibration process using a calibration feature on a mouthpiece, according to one embodiment. The physical mouthpiece assembly 401 has one or more calibration design features 901 to calibrate the instrument assembly 501 with respect to the physical mouthpiece assembly 401. The coordinates of the design features 901 are known to the planning software. When the drill bit 503 is inserted into an implant site, the planning software 201 checks if the tracked drill position 902 matches to the predefined position 903.

Multiple calibration features may be implemented over the mouthpiece and are strategically spread along the mouthpiece in order to achieve the accuracy of calibration and reduce the calibration error. If the position 902 and 903 do not match, the planning software 201 re-calibrates the tracking components based on the reading of the position information to compensate for the error. The recalibration method may vary depending on the type of error and transmitter and sensor setup. According to one embodiment, the positions of the mouthpiece assembly 401 and the instrument assembly 601 are calibrated within the planning software with respect to a reference point (e.g., calibration design features 901). When the physical instrument assembly 501 is positioned and oriented in a known position and angle, the calibration error is calculated in planning software 201. After the calibration error calculation is complete, the correction is applied and the virtual instrument assembly and mouthpiece assembly are updated accordingly. The calibration reset maybe signaled back to the doctor visually, audibly, and via a tactile feedback, or any other type of feedback.

A magnetic-based navigation system in dental implant surgery has been disclosed. Although various embodiments have been described with respect to specific examples and subsystems, it will be apparent to those of ordinary skill in the art that the concepts disclosed herein are not limited to these specific examples or subsystems but extends to other embodiments as well. Included within the scope of these concepts are all of these other embodiments as specified in the claims that follow.

What is claimed is:

1. A surgical navigation system comprising:
a custom piece made to fit in a patient's anatomy, the custom piece comprising a first magnetic component;
an instrument assembly comprising a drill bit and a second magnetic component;
a processing unit receiving at least one of a first position and orientation data from the first magnetic component and a second position and orientation data from the second magnetic component; and
a display displaying the at least one of the first position and orientation data of the first magnetic component and the second position and orientation data of the second magnetic component.

2. The surgical navigation system of claim 1, wherein the first magnetic component is a magnetic transmitter, and the second magnetic component is a magnetic sensor.

3. The surgical navigation system of claim 2, wherein the magnetic sensor receives signal transmitted from the magnetic transmitter and determines the second position and orientation data based on the received signal from the magnetic transmitter.

4. The surgical navigation system of claim 1, wherein the first magnetic component is a magnetic sensor, and the second magnetic component is a magnetic transmitter.

5. The surgical navigation system of claim 1 further comprising a magnetic transmitter, wherein the first magnetic component and the second magnetic component are magnetic sensors receiving signals transmitted from the magnetic transmitter.

6. The surgical navigation system of claim 1, wherein the custom piece is fabricated based on a virtual piece created from a CT scan or a dentition surface model of the patient using a planning software.

7. The surgical navigation system of claim 6, wherein the dentition surface model is generated from a CT scan of a patient's impression, an optical scan of a patient's impression, a stone model, an intra-oral scan, or any combination thereof.

8. The surgical navigation system of claim 1, wherein the custom piece is fabricated with an intra-oral scan data of the patient merged to the CT scan data of the patient.

9. The surgical navigation system of claim 1, wherein the processing unit generates a warning sign of unreliable tracking or an occurrence of magnetic field interference of at least one of the first magnetic component and the second magnetic component.

10. The surgical navigation system of claim 9, wherein the warning sign is one of an audible feedback, a visual feedback, and a haptic feedback.

11. The surgical navigation system of claim 1 further comprising a registration apparatus for registering an offset of the drill bit, wherein the offset is measured from the second magnetic component to a tip of the drill bit.

12. The surgical navigation system of claim 1, wherein the custom piece further comprises a calibration feature to calibrate the instrument assembly with respect to the custom piece.

13. A method comprising:
receiving a first position and orientation data of a first magnetic component;
processing the first position and orientation data of the first magnetic component with respect to a second position and orientation data of a second magnetic component;
tracking the first position and orientation of the first magnetic component with respect to the second magnetic component.

14. The method of claim 13, wherein the first magnetic component is integrated with a custom piece made to fit in a patient's anatomy and the second magnetic component is integrated with an instrument assembly comprising a drill bit.

15. The method of claim 13, wherein the first magnetic component is a magnetic transmitter, and the second magnetic component is a magnetic sensor.

16. The method of claim 14, wherein the magnetic sensor receives signal transmitted from the magnetic transmitter and determines the second position and orientation data based on the received signal from the magnetic transmitter.

17. The method of claim 13, wherein the first magnetic component is a magnetic sensor, and the second magnetic component is a magnetic transmitter.

18. The method of claim 13, wherein the first magnetic component and the second magnetic component are magnetic sensors receiving signals transmitted from a magnetic transmitter.

19. The method of claim 13, wherein the custom piece is fabricated based on a virtual piece created from a CT scan or a dentition surface model of the patient using a planning software.

20. The method of claim 13 further comprising generating a warning sign of unreliable tracking or an occurrence of magnetic field interference of at least one of the first magnetic component and the second magnetic component.

21. The method of claim 20, wherein the warning sign is one of an audible feedback, a visual feedback, and a haptic feedback.

22. The method of claim 13 further comprising registering an offset of the drill bit, wherein the offset is measured from the second magnetic component to a tip of the drill bit.

23. The method of claim 13 further comprising providing at least one calibration feature to calibrate the instrument assembly with respect to the custom piece.
